(19) United States
(12) Patent Application Publication
(10) Pub. No.: US 2020/0016396 A1
(43) Pub. Date: Jan. 16, 2020

(54) SYSTEMS AND METHODS FOR IMPROVING PLACEMENT OF DEVICES FOR NEURAL STIMULATION

(71) Applicant: Synchron Australia Pty Limited, Parkville VIC (AU)

(72) Inventor: Peter Eli YOO, Fitzroy (AU)

(73) Assignee: Synchron Australia Pty Limited, Parkville VIC (AU)

(21) Appl. No.: 16/509,403

(22) Filed: Jul. 11, 2019

Related U.S. Application Data
(60) Provisional application No. 62/696,628, filed on Jul. 11, 2018.

(51) Int. Cl. A61N 1/05; A61B 90/00; A61B 34/10; A61N 1/36

(52) U.S. Cl. CPC A61N 1/0534; A61B 90/37; A61B 34/10; A61B 2090/374; A61B 2090/364; A61N 1/0531; A61B 2090/3762; A61N 1/3605

(57) ABSTRACT

The present invention involves systems and methods for creating and displaying surgical plans for implanting devices in the body.

### RELATED APPLICATION

This is a non-provisional application of U.S. Provisional application No. 62/696,628 filed on Jul. 11, 2018, the entirety of which is incorporated by reference.

### BACKGROUND

Signal generating and/or receiving devices can be implanted in or on the body to stimulate tissue, record signals from tissue, or both. For example, electrodes can be implanted in or on the body to stimulate tissue, record signals from tissue, or both.

Implanting electrodes in or near a desired (e.g., ideal) cortical location can influence the efficacy of stimulating and/or recording neural tissue (e.g., using brain-computer-interfaces) such as decoding thoughts from neural signals. Sub-optimal placement of recording devices (e.g., electrode arrays) can lead to recording of noise, and thus decoding inaccuracies. Sub-optimal placement of stimulating devices (e.g., electrode arrays) can also lead to sub-optimal or ineffective stimulation of neural tissue. This is especially true, for example, for endovascular electrode array implantations, as such devices are deployed within the segment of the blood vessel that lies near (e.g., as close as possible) to the desired cortical location.

Neuroendovascular surgeries are often radiologically guided, for example, with intra-operative magnetic resonance imaging as well as with intra-operative computed tomography (CT), where the interventionist can view the outline of the vessels, but such surgical techniques do not currently provide the interventionist with static or real-time functional and/or physical reference marks for determining whether a current position of a recording and/or stimulating device (e.g., an electrode array) within a vessel is near the preferred target cortical location prior to, during, or after deployment of the device. Physical references alone such as vessel tortuosity, vessel size, aneurism location and blockage locations currently do not allow interventionists to determine whether the position of the device is in the preferred target cortical location, as such physical references are currently not associated with or linked to any functional activity. Conventional radiologically guided methods (e.g., CT methods) do not show any static or real-time functional activity. Further, whether the cortical location adjacent to the current device position inside the vessel is the desired cortical location for thought-decoding cannot currently be determined from within the vessel, as the cortex is not exposed at all during the minimally invasive endovascular surgery and cannot be stimulated intraoperatively, which is the status quo of open-brain surgery.

Therefore, there remains a need to address the problems with device implantation and/or to improve current electrode effectiveness, including, for example, (1) identifying the desired implantation location within the vessel in relation to the target cortical location and (2) electrophysiologically confirming the target cortical location by stimulation from within the vessel. The following disclosure describes these and other advantages and improvements to neural stimulation and/or recording.

The following disclosure provides examples and any variation of these examples are within the scope of the disclosure. Such variations can include any combination of embodiments or any combination of aspects of embodiments wherever possible. Further, the following description can be combined with conventional DBS. Other features, advantages, and variations of the invention will be apparent to those skilled in the art from the following description and accompanying drawings, wherein, for purposes of illustration only, specific forms of various examples are set forth in detail.

### BRIEF SUMMARY

Systems and methods are disclosed for implanting electrodes in blood vessels in and/or outside the brain.

More generally, systems and methods are disclosed for implanting endovascular devices in blood vessels in and/or outside the brain. For example, a method under the present disclosure includes implanting a medical device within a vessel in a region of a patient's body, where the device is configured for sensing or stimulating tissue adjacent to the vessel.

One variation of the method can include obtaining an activity image of the region of the patient's body, where the activity image displays a neural activity in the region; obtaining a structural image of the region of the patient's body, where the structural image displays an anatomic structure of a vascular network in the region, where the vessel is part of the vascular network; co-registering the activity image with the structural image to produce a composite image of the region showing the neural activity displayed in the activity image relative to the anatomic structure of the vascular network of the structural image; identifying a land-mark based target corresponding to the neural activity; acquiring a real-time image of the region of the patient's body; co-registering the composite image with the real-time image to select a target location within the vascular network using the land-mark based target; and implanting a device within the target location, where the device is configure d to sense and/or stimulate neural activity in the region.

In one variation of the method, obtaining the activity image comprises performing at least a first non-invasive imaging of the region. An additional variation includes where the first non-invasive imaging of the region comprises an imaging modality selected from the group consisting of functional magnetic resonance imaging (fMRI), MRV, electroencephalogram (EEG), magnetoencephalography (MEG), functional near-infrared spectroscopy (fNIRS), functional positron emission tomography (PET), and any combination thereof.

Another variation of the method can include obtaining the structural image by performing at least a second non-invasive imaging of the region. Additional variations include where the second non-invasive imaging comprises an imaging modality selected from the group consisting of: structural magnetic resonance imaging (sMRI), magnetic resonance venography (MRV), computed tomography (CT), and any combination thereof

The method can further comprise identifying the target location with a virtual marker and displaying the virtual marker on the real-time three-dimensional image.

In an additional variation, the method includes identifying a landmark based target corresponding to the neural activity comprises identifying a plurality of landmark based targets. The landmark based target can comprise a target selected from the group consisting of a target location, a deployment location, a structural landmark, a functional landmark, a structural and functional landmark, a problem area, and any combination thereof

A variation of the method includes comprising immobilizing the region of the patient's body prior to acquiring the real-time three-dimensional image.

The images described herein can comprise three-dimensional images and/or two dimensional images.

Implanting the device within the target location can comprise advancing the device through the vascular network of the patient.

Variations of the method can include co-registering the activity image with the structural image by overlaying the activity image with the structural image. Moreover, co-registering the activity image with the structural image further can comprise displaying the activity image and the structural image separately side-by-side.

Co-registering the composite image with the real-time three-dimensional image can include selecting the target location within the vascular network using the landmark based target comprises displaying a virtual representation of the landmark based target on the real-time image.

Another variation of the method includes a method for providing guidance for implanting a medical device within a vessel in a region of a patient's body. For example, the method can include obtaining an activity image of the region of the patient's body, where the activity image displays a neural activity of non-vascular tissue in the region; obtaining a structural image of the region of the patient's body, where the structural image displays an anatomic structure of a vascular network in the region; co-registering the activity image with the structural image to produce a composite image of the region showing the neural activity displayed in the activity image relative to the anatomic structure of the vascular network of the structural image to identify a land-mark based target; acquiring a real-time three-dimensional image of the region of the patient's body; co-registering the composite image with the real-time three-dimensional image such that the land-mark based target can be identified using the real-time three-dimensional image to provide an operative map to a medical practitioner such that the medical practitioner is able to use the landmark based target to identify an implant location for implantation of the medical device in the region of the patient's body.

### BRIEF DESCRIPTION OF DRAWINGS

The drawings shown and described are exemplary embodiments and non-limiting. Like reference numerals indicate identical or functionally equivalent features throughout.

FIG. 1 illustrates a conventional deep brain stimulation device containing electrodes that are implanted within a brain of an individual.

FIG. 2A illustrates an example of a method as described herein with pre-operative and intraoperative steps.

FIG. 2B illustrates a variation of a method described herein shown in a flow-diagram illustrating pre-operation/planning and intra-operation/operative portions of the method.

FIG. 2C illustrates a variation of the pre-operation portion of FIG. 2B.

FIG. 2D illustrates a variation of the intra-operation method of FIG. 2B.

FIG. 2E illustrates a variation of the intra-operation method of FIGS. 2B and 2D.

FIGS. 3A-3C illustrates a representation of pre-operative images with operative images.

FIGS. 4A-4C illustrates a representation of pre-operative images with operative images having one or more virtual markers displayed on the operative image.

FIG. 5A-5C illustrates a representation of pre-operative images with operative images having one or more virtual markers displayed on the operative image

FIGS. 6A-6C illustrates an example of observing an implant being delivered to a target area using a real-time image of the target location where the real-time image also includes a virtual marker.

FIGS. 7A-7C illustrates another example of pre-operation/planning images being displayed with real time imaging.

FIGS. 8A-8C illustrates another example of pre-operation/planning images being displayed with real time imaging.

FIGS. 9A-9C illustrate an example of real-time images of an implant being advanced towards a target location.

FIGS. 10A-10C illustrate another example of real-time images of an implant being advanced towards a target location.

### DETAILED DESCRIPTION

This disclosure is not limited to the particular embodiments, variations, or examples described, as such may, of course, vary. For example, while neural tissue in the brain is referred to throughout the disclosure, the disclosure is applicable to any tissue that emits signals that can be recorded and/or has cells which can be stimulated. This can include tissue and vessels anywhere in the body, for example, the brain, the neck, extremities, and torso.

Structural and functional imaging techniques can be used to design a surgical plan, where the surgical plan can have one or multiple target locations for a device, for example, 1 to 5 or more target locations, including every 1 target location increment within this range (e.g., 1 target location, 2 target locations, 3 target locations, . . . , 5 target locations). Clearly, the number of target locations can vary depending upon the intended application. The target locations can be ranked from most preferred to least preferred, can be ranked equally such that each is equally preferred, can be non-ranked, or any combination thereof (e.g., a primary location and zero or multiple secondary locations). The target locations can be in the same or different vessel as another target location. For example, a first target location can be in a first vessel and a second target location can be in the first vessel, a second vessel, or both vessels. Multiple target locations can advantageously provide backup options should complications during surgery. The surgical plan can be displayed on a display during surgery so that functional data, structural data, the one or more target locations, or any combination thereof can be visually displayed before and during surgery.

One or more structural imaging techniques can be used to design the surgical plan. The structural imaging techniques can include, for example, structural magnetic resonance imaging (sMRI), magnetic resonance venography (MRV), computed tomography (CT), or any combination thereof.

One or more functional imaging techniques can be used to design the surgical plan. The functional imaging techniques can include, for example, functional magnetic resonance imaging (fMRI), MRV, electroencephalogram (EEG), magnetoencephalography (MEG), functional near-infrared spectroscopy (fNIRS), functional positron emission tomography (PET), or any combination thereof.

In designing a surgical plan, one or more structural imaging techniques can be used, one or more functional imaging techniques can be used, or both types of imaging techniques can be used. One or multiple pre-surgical scans and images can be acquired before a surgery to generate a surgical plan.

For example, pre-surgical magnetic resonance imaging (MRI) can be used to investigate the cortical areas that functionally correspond to a given thought or behaviour (e.g., using fMRI and/or sMRI), to investigate the morphological details of the vasculature nearby the cortical areas (e.g., using MRV), or both. The results can provide 3D images with the information of estimated target cortical location according to functionality in relation to the potential desired (e.g., ideal) implantation location within the vessel, as well as the structural details of the nearby vessels. This information can be merged together to form a functional activation map that shows the likely cortical area that gives rise to the given thought or behaviour and further, which specific blood vessel lies nearby for device implantation for pre-surgical planning, intra-operative use, or both. The pre-surgical MRI data can include fMRI, sMRI, MRV, or any combination thereof.

The resulting MRI images can be uploaded to an intra-operative computed tomography (CT) scanner. The CT scanner can be used for radiologically guided neuroendovascular surgery. While the patient's head is fixed to a specific position, a CT image of the patient's head can be acquired. Then, the uploaded pre-surgical MRI images can be co-registered to the CT image acquired. The co-registration can be a 2D co-registration, a 3D co-registration, or both. The co-registration can transform the pre-surgical MRI images into the patient's current head position (as it is now fixed; i.e., “real-time space”). The uploaded and co-registered images can be sliced (2D views) in any direction according to the CT scanner arm position, and/or be used as underlays and/or overlays for guiding the implantation procedure. This advantageously allows the interventionists to see the functional target cortical location in relation to the current device (e.g., electrode array) position within the vessel. If necessary, and if the patient is healthy enough to be awake during surgery, adjacent cortical areas can be stimulated with the device during surgery to confirm that the current segment of the vessel is located adjacent to the target cortical location.

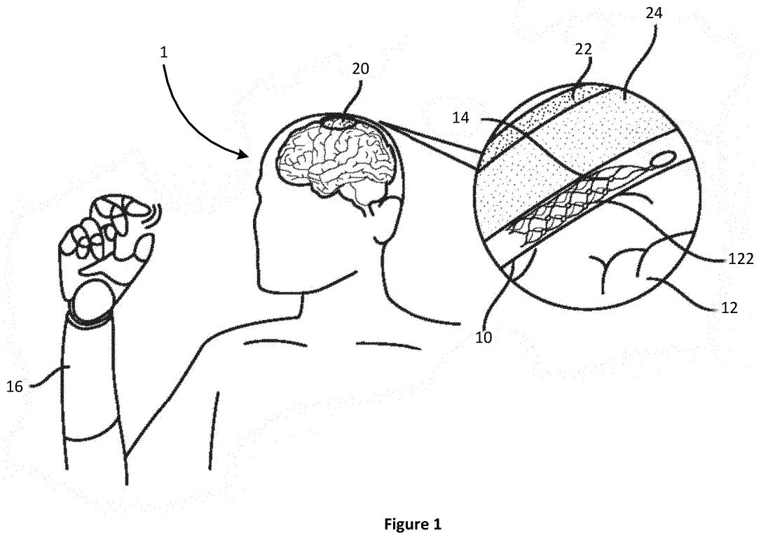

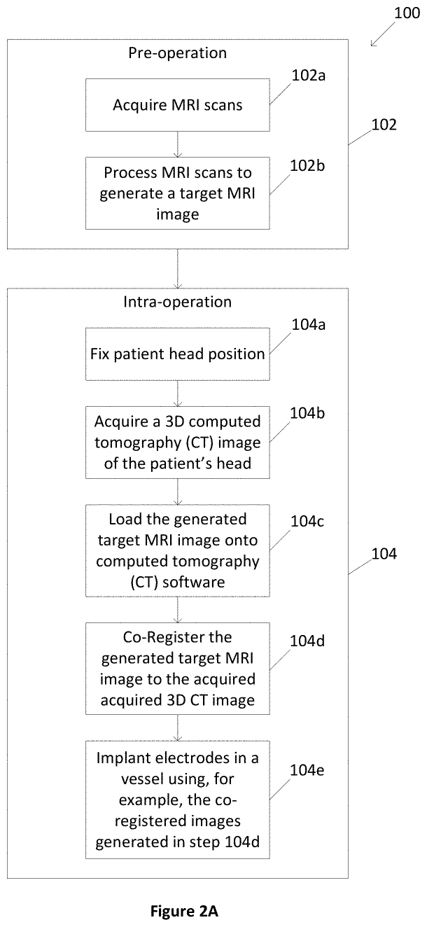

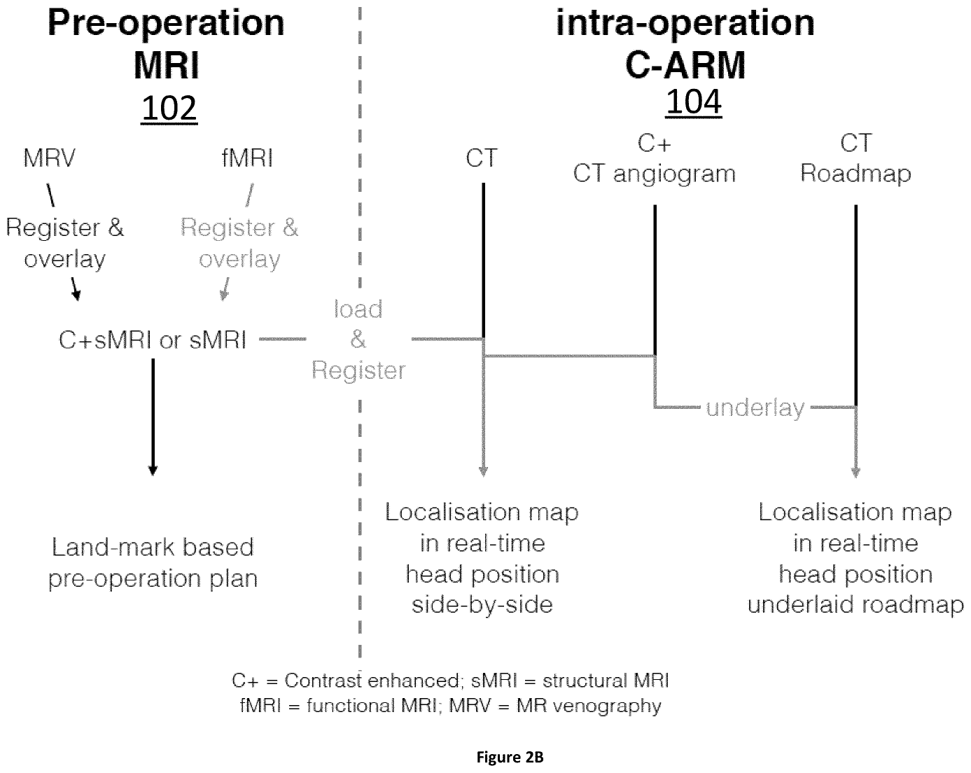

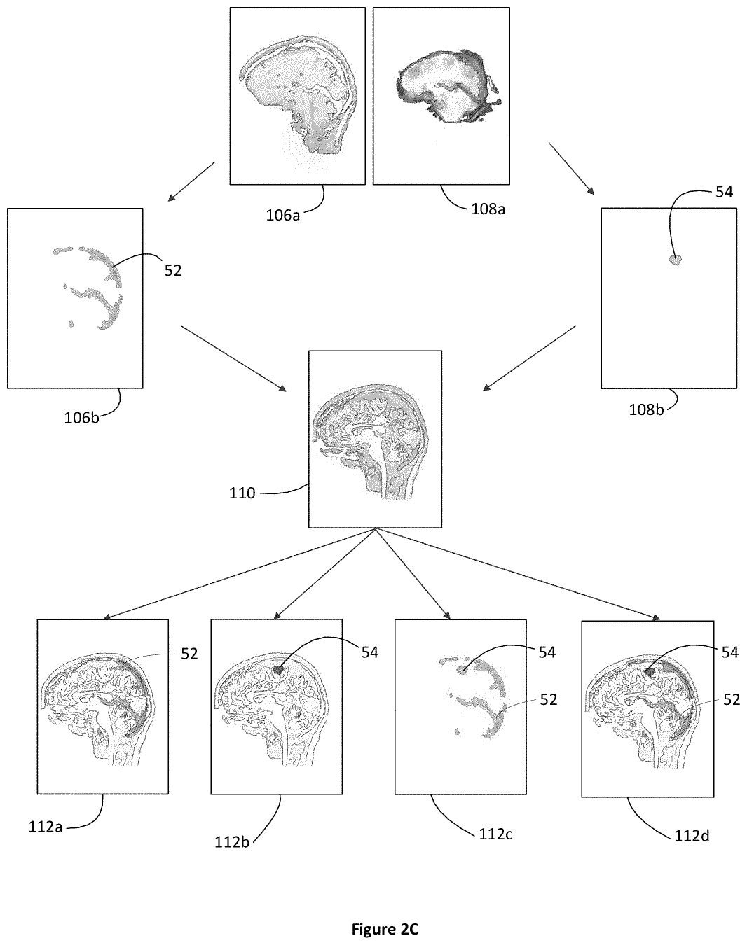

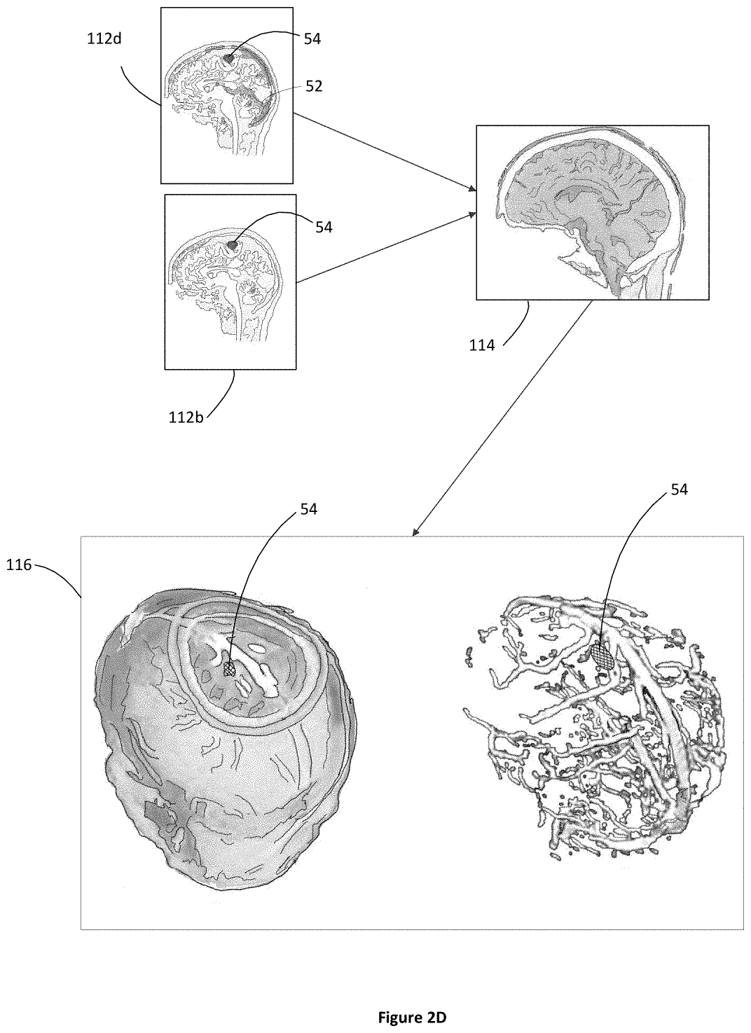

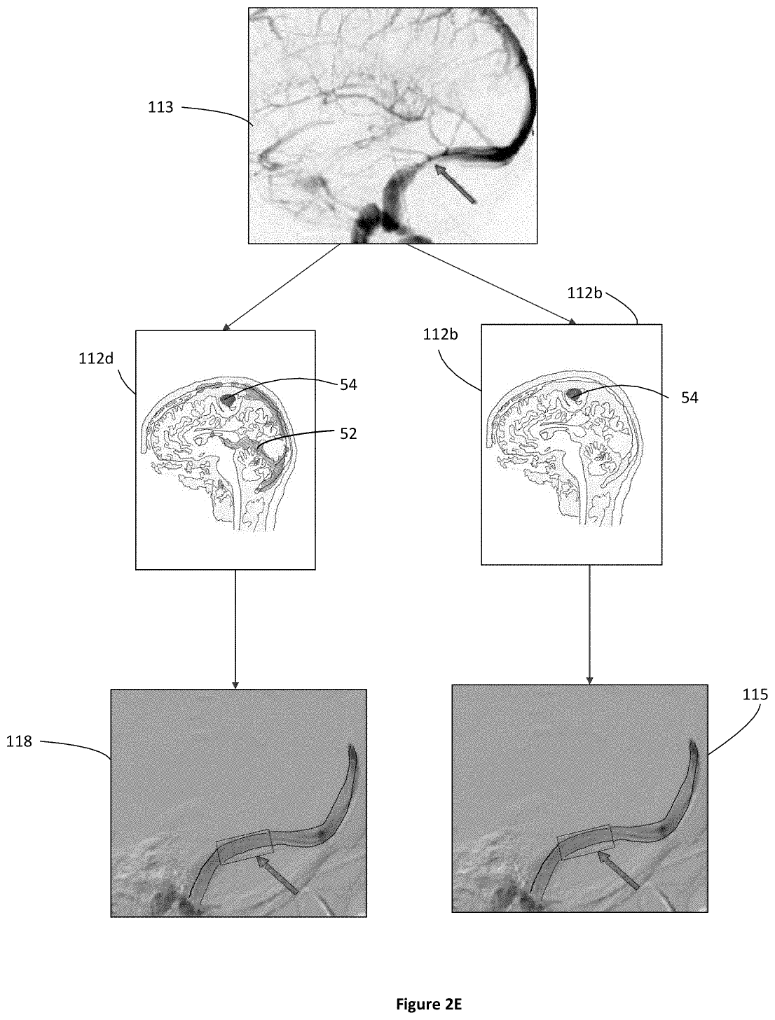

FIG. 1 illustrates a variation of a of an implant a device (e.g., electrode array) in a vessel. As shown by FIG. 2A, the method 100 can include one or more pre-operation or planning steps 102 and one or more intra-operation/operative steps 104.

FIG. 2A further illustrates that the pre-operation step 102 can include an MRI acquisition step 102a. Step 102a can include, for example, performing magnetic resonance venography (MRV) to acquire an enhanced image of the venous vasculature, performing task functional magnetic resonance imaging (fMRI) to acquire one or multiple blood-oxygenation-level-dependent (BOLD) activation locations, performing structural magnetic resonance imaging (sMRI) to acquire high-resolution anatomical details of the head and brain, or any combination thereof.

FIG. 1 illustrates one application of an implant 122 that functions as a neural interface. Such an implant 122 can have any number of uses, but in the illustrated variation, the implant 122 is positioned within a vessel 10 that is part of a vascular network within the patient 1. The implant 122 includes any number of electrodes 14 that transmits neural activity from neural tissue 12 so that the patient 1 can communicate/control an external device 16 (e.g., a prosthetic arm). Accordingly, the implant 122 must be placed in an area 20 that generates the neural activity intended to be received by the implant 122. Therefore, it is important that positioning of the implant 122 during the operative procedure permits placing the implant at a pre-determined area where the desired neural activity occurs. Moreover, because the implant 122 can be positioned using a vascular approach, positioning of the implant 122 occurs without opening the skin 22 or bone (skull) 24) of the patient 1.

FIG. 1 illustrates the implant 122 within a blood vessel 10 overlying the motor cortex in the patient 1, which allows it to sense neural-related signals. The implant can be wired or wireless and can optionally communicate with one or more host devices (not shown).

The methods of the present disclosure can be combined with the devices disclosed in commonly related patent application Ser. No. 14/348,863 filed Mar. 31, 2014 (publication no. US 20140288667); Ser. No. 16/164,482 filed Oct. 18, 2018 (publication no. US20190046119); Ser. No. 15/957,574 filed Apr. 19, 2018 (publication no. US20180236221); Ser. No. 15/955,412 filed Apr. 17, 2018 (publication no. US20180303595); Ser. No. 16/054,657 filed Aug. 3, 2018 (publication no. US20190038438); Ser. No. 16/405,798 filed May 7, 2019; and Ser. No. 16/457,493 filed Jun. 28, 2019. The entirety of each of which are incorporated by reference herein.

FIG. 2A further illustrates that the pre-operation step 102 can include an MRI processing step 102b. After the pre-operation MRI scans are acquired in step 102a or otherwise obtained, step 102b can include, for example, extracting veins from the MRV to form a venogram, extracting areas of blood-oxygenation-level-dependent (BOLD) activation locations from the fMRI data to form a BOLD activation map, registering MRV and fMRI to sMRI, transforming the venogram and BOLD activation map to sMRI space, superimposing the transformed BOLD activation map and/or venogram onto the sMRI, or any combination thereof. Step 102b can result in a 2D or 3D image that shows the target location in relation to a patient's anatomical details, including the vasculature. This resulting 2D or 3D image is also referred to as a target MRI image.

FIG. 2A further illustrates that the intra-operation step 104 can include, for example, fixing the patient head position in step 104a, acquiring a 3D image of patient's head using planar CT in step 104b, loading the target MRI image onto CT machine software in step 104c, co-registering the target MRI image to the CT image acquired in step 104d, implanting a device (e.g., electrodes) in a vessel using any of the acquired, generated, and/or co-registered images, or any combination of these steps. The co-registering step 104c can transform the target MRI image into real-time space, and thus create a target MRI image that can be sliced in any direction according to the acquisition direction of intra-operation CT imaging. The vessel can be any vessel in the brain or outside the brain. Step 104d advantageously allows interventionists to visually see the one or multiple target locations in relation to the vasculature in real-time while the device is being inserted to a deployment location and while the device is being deployed at the deployment location during implantation step 104e. The deployment location can be the same or different as the target location. For example, the target locations can correspond to the device deployment location or to a region adjacent the deployment location that the device is intended to record and/or stimulate while deployed in the deployment location. The surgical plan can have one or more target locations, one or more deployment locations as described above with reference to the one or more target locations, or both types of locations.

The method 100 can involve repeating and performing operations 102 and 104 or any combination thereof.

The operations 102, 102a, 102b, 104, 104a, 104b, 104c, 104d and 104e can be interchangeably combined, rearranged, substituted, and/or omitted in any combination, and can be executed in any order, for example, in the order shown in FIG. 2A. Additional operations that are not shown can be added to the method 100 or can be part of a separate implementable and/or performable method, for example, implanting electrodes to one or more other target locations.

FIG. 2B illustrates a variation of a flow diagram for the pre-operation and intra-operation processes 102, 104.

FIG. 2C illustrates a variation of a pre-operation step 102 (as shown in FIGS. 2A and 2B) that can be used to generate one or more landmark based pre-operation plans (e.g., plans 112a-112d) that can optionally display virtual images 52, 54 based on landmarks that are identified in the imaging process. The landmarks can be functional landmarks, structural landmarks, or both. For example, image 106a represents an MRV image, image 106b represents an image of the veins 52 extracted from image 106a, image 108a is an fMRI image, image 108b is an image of an activation map calculated from image 108a to show a region of neural activity 52, image 110 is a sMRI image that can be derived from the previous processing. The images can be co-registered to allow for display of any of images 112a, 112b, 112c, and/or 112d. For example, image 112a is an image showing a virtual representation 52 of the extracted veins in image 106b registered and overlaid with the sMRI image 110, image 112b is an image of the calculated activation map with a virtual representation of neural activity 52 from image 108b registered and overlaid with the sMRI image 110, image 112c is an image of the extracted veins in image 106b registered and overlaid with the calculated activation map in image 108b to show the virtual representation of the veins 52 and virtual activity region 54, and image 112d is an image of the extracted veins 52 in image 106b and the calculated activation map with activity region 54 in image 108b registered and overlaid with the sMRI image 110. FIG. 2C illustrates that the MRV, extracted veins from the MRV, fMRI, the calculated activation map from the fMRI and sMRI can be overlaid with one another in any combination, for example, the combinations illustrated in images 112a, 112b, 112c and 112d. It should be noted that the images described herein can comprise 2 dimensional images or 3 dimensional images/models.

FIG. 2D illustrates a variation of an intra-operation CT image 114 that can be a live image. In this example, any of the co-registered images 112a, 112b, 112c, and/or 112d can be co-registered with a live CT image 114 to produce a real-time virtual representation of the information from the CT image 114 with the veins 52 and/or neural activity region 52 registered and overlaid onto the CT image 114. The resulting 3d image 116 can be sliced in any direction according to the position of the scanner in real time as well as display a virtual (or real) representation of the veins and/or region of neural activity 52.

FIG. 2E illustrates that the conventional approach does not use a functional target overlaid or underlaid onto the CT image 113 (e.g., a magnified viewing field of image 114), an angiogram image, or their combination as indicated by the word “None” in FIG. 2D.

FIG. 2E further illustrates that any combination of the sMRI, fMRI, and MRV can be over- and/or under-laid with one another and be displayed to the interventionist, for example, side-by-side with a real-time CT image (and/or with an angiogram) e.g., displaying image 112b (structural and functional MRI), 112d (sMRI, fMRI & MRV) and 114 side-by-side or over- and/or underlaid onto the real-time CT image (and/or angiogram) (e.g., image 116 having any combination of images). For example, image 115 shows a magnified view of the activation map calculated from image 108a registered and overlaid onto the CT image 114. As another example, image 118 shows a magnified view of image 116. The side-by-side or co-registered overlaid/underlaid images advantageously allow interventionists to identify the target implantation location within the vessel in relation to the target cortical location. The interventionist can electrophysiologically confirm the target cortical location by stimulation from within the vessel.

One or more pre-surgical images (e.g., images 112a-112b) can be combined with one or more surgical images (e.g., images 113, 114), for example, to generate images 115, 116, 118. Any of the images disclosed can be used to identify the desired implantation location within the vessel in relation to the target cortical location.

As a first example, a target MRI image (e.g., one or more of the images 112a-112d) can be used as an underlay (or overlay) and superimposed on the CT road-map (e.g., real-time image 114) to guide electrode delivery, or any combination of these steps or other steps disclosed herein.

As a second example, the interventionist can perform an intra-operation contrast-enhanced angiogram, superimpose the CT road-map (e.g., real-time image 114) onto the angiogram, and use a target MRI image (e.g., one or more of the images 112a-112d) side-by-side with the superimposed roadmap to guide electrode delivery, or any combination of these steps or other steps disclosed herein.

As a third example, the interventionist can perform an intra-operation contrast-enhanced angiogram, superimpose the angiogram onto a target MRI image (e.g., one or more of the images 112a-112d), use the resulting image as an underlay (or overlay), and superimpose the CT road-map (e.g., real-time image 114) onto (or under) the resulting image to guide electrode delivery, or any combination of these steps or other steps disclosed herein.

As a fourth example, the interventionist can perform an intra-operation contrast-enhanced angiogram, superimpose the angiogram onto a target MRI image (e.g., one or more of the images 112a-112d), use the resulting image side-by-side with a roadmap superimposed onto angiogram to guide electrode delivery, acquire CT image of the patient head on table using intra-operative angio/CT scanner, load the activation map image onto the angio/CT scanner, co-register the MRI to CT—(built-in function of the angio/CT machine), and perform angiogram/roadmap and superimpose onto the co-registered MRI image, or any combination of these steps or other steps disclosed herein.

Any two or more images can be displayed side-by-side, overlaid with one another, underlaid with one another, or any combination thereof. When displayed side by side, the images can be displayed on one or multiple displays. For example, each image can be displayed on a separate display. The term image refers to both static and real-time images.

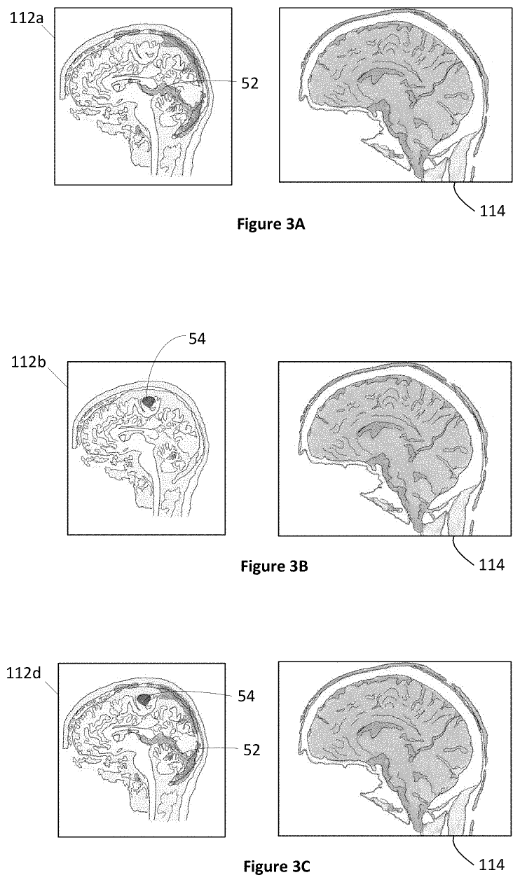

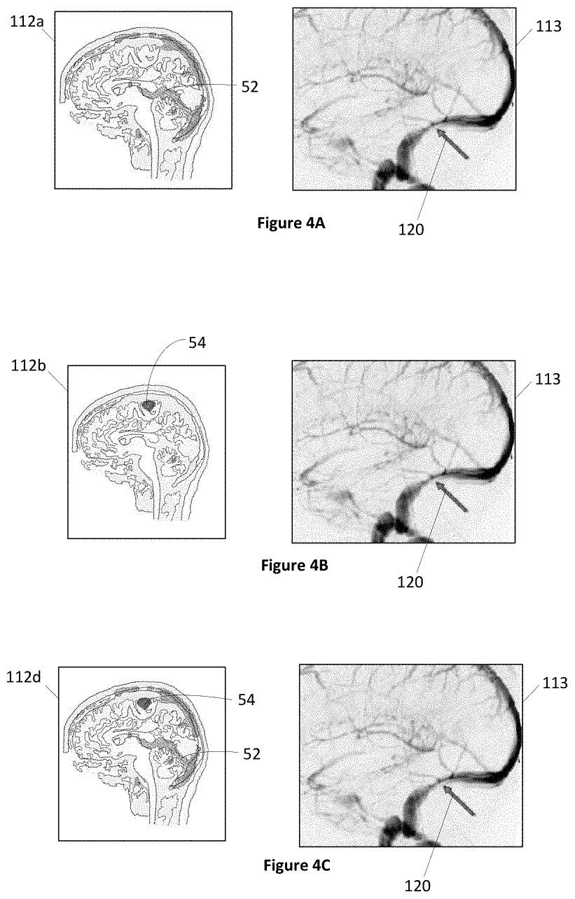

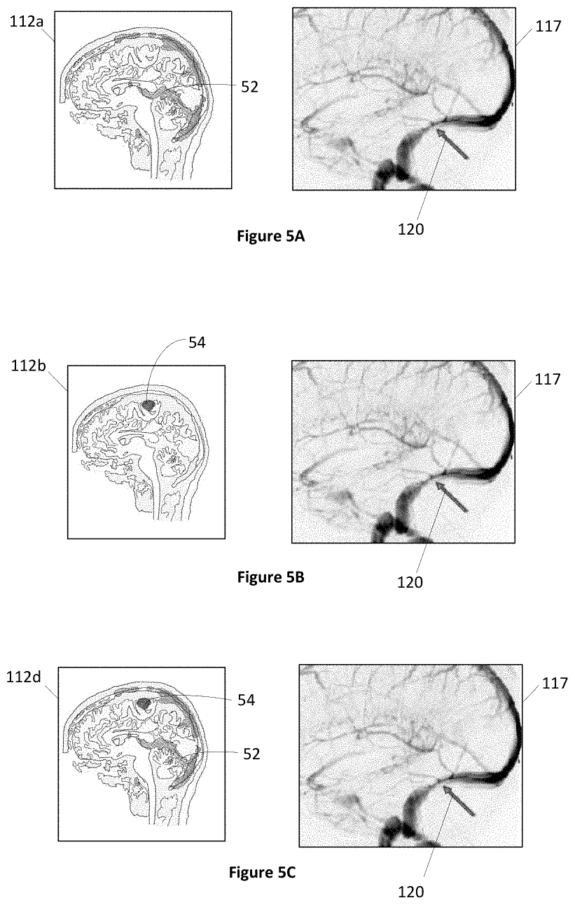

For example, FIGS. 3A-5C illustrate various images displayed side-by-side with one another. Image 117 in FIGS. 5A-5C is a magnified area of an angiogram. FIGS. 4A-5C further illustrate that the system can display one or more markers 120 on any of the images described, illustrated, and/or contemplated herein. The marker 120 can indicate a target location, a deployment location, a structural landmark, a functional landmark, a structural and functional landmark, a problem area (e.g., an area with stenosis or an aneurysm), or any combination thereof. The markers 120 can identify functional and structural attributes (e.g., blood flow, electrical activity) that are visible or not visible on the displayed or combined images. For example, the marker 120 can be shown on a CT image but be derived from another image such as an angiogram. Although a marker 120 is illustrated in FIGS. 4A-5C, these images can be displayed with or without markers 120. The interventionist can identify the target location, the deployment location, the structural landmark, the functional landmark, the structural and functional landmark and problem areas using the displayed images without markers (e.g., markers 120), or with the help of one or multiple markers 120.

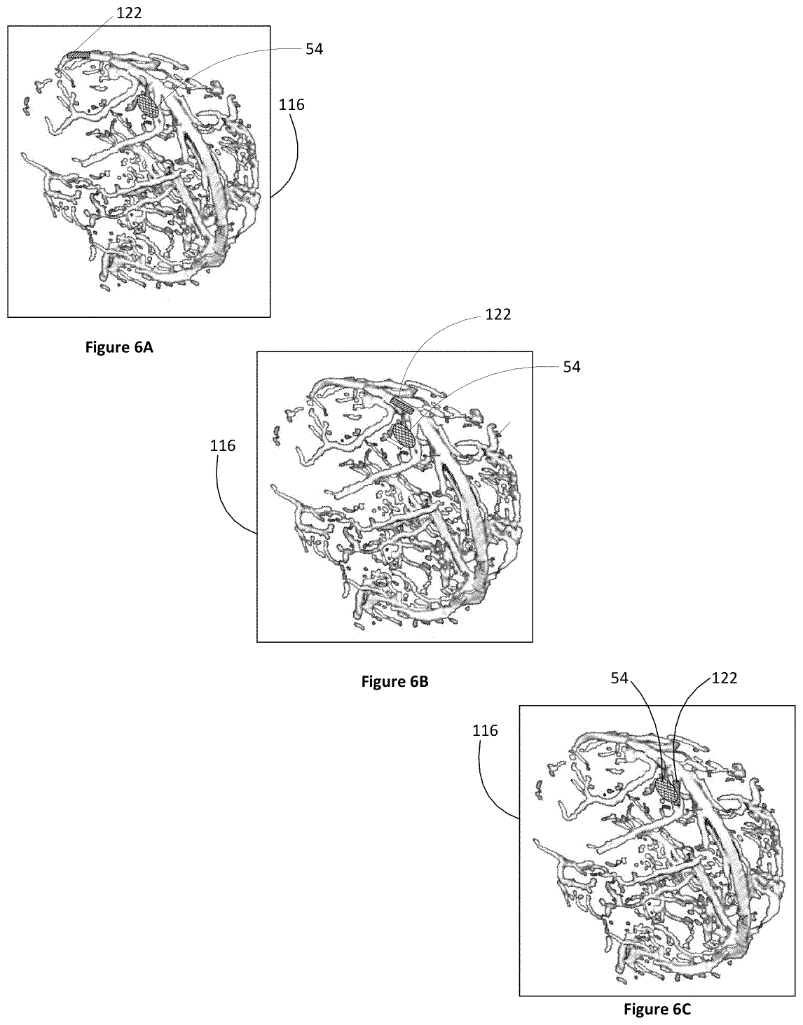

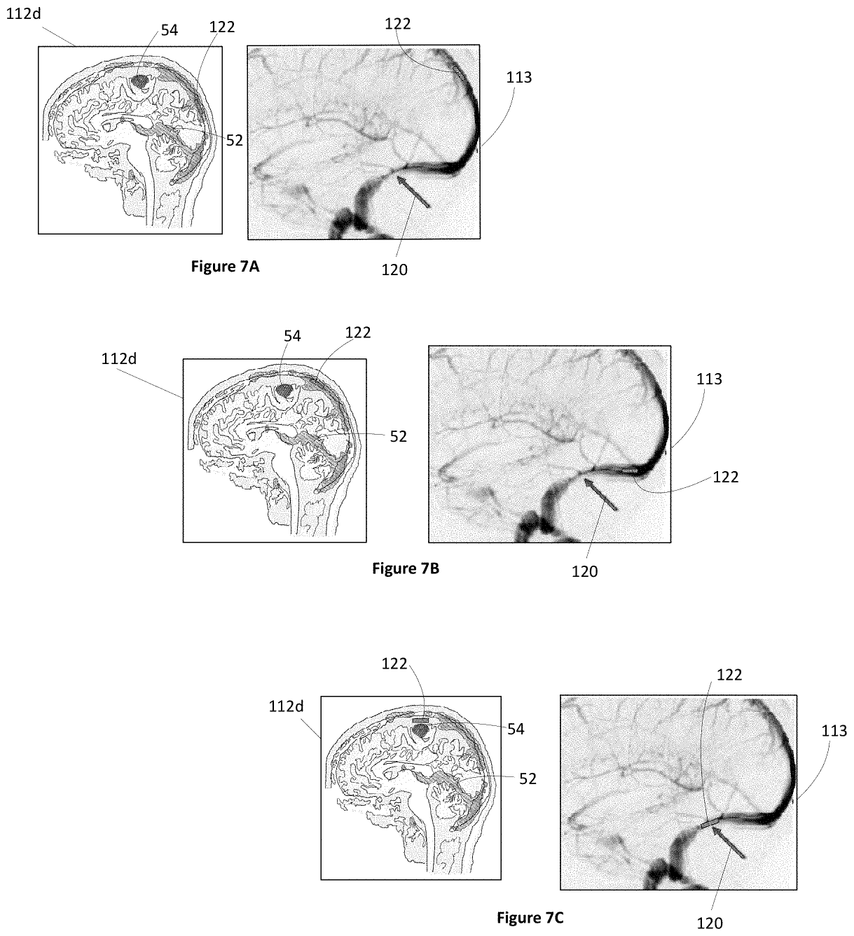

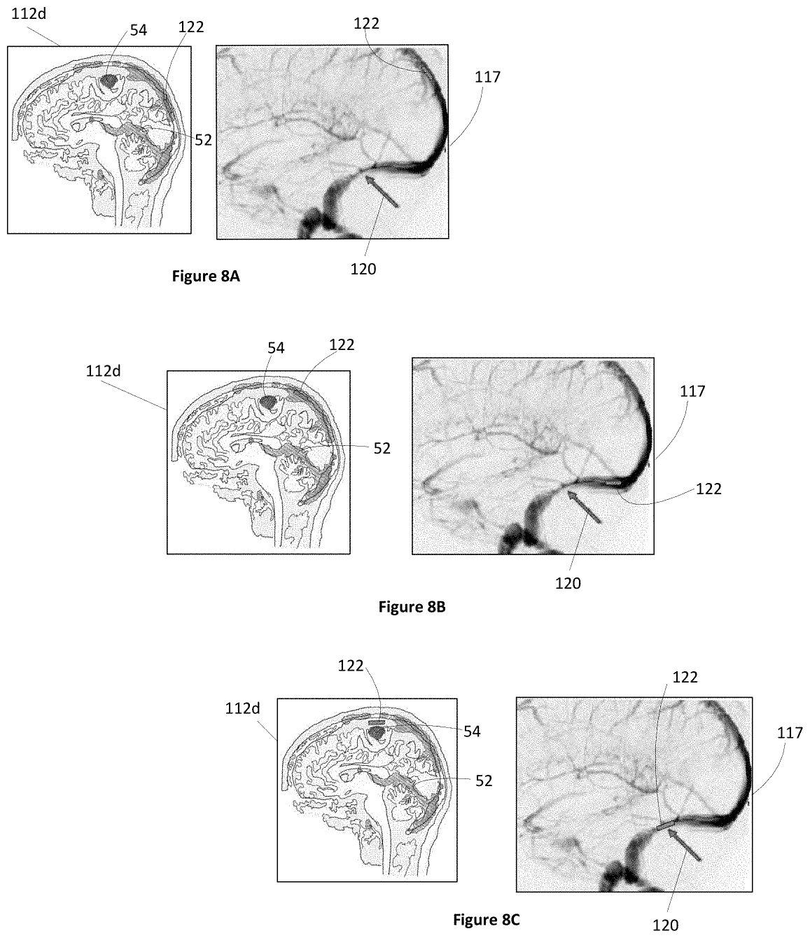

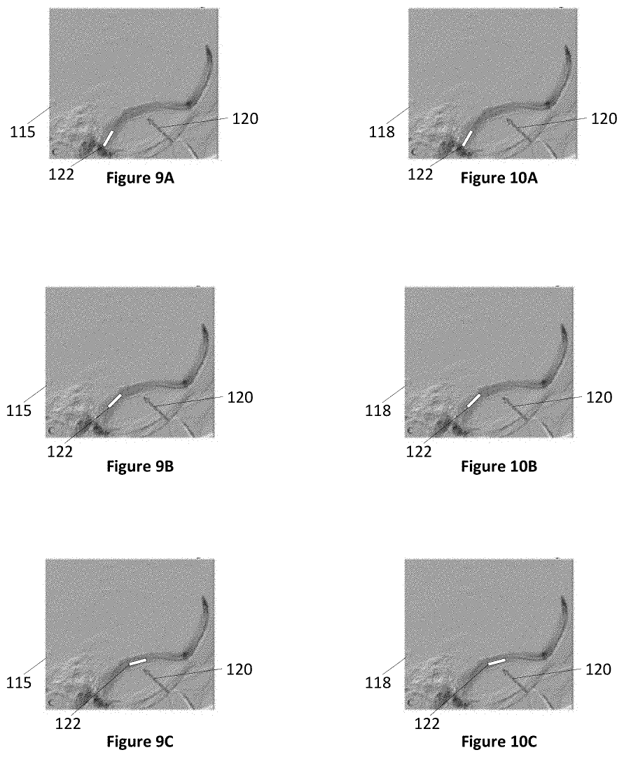

The images can be displayed to an interventionist (e.g., a surgeon) while implanting an implant 122. The implant 122 can be an endovascular device, for example, an endovascular stent. The stent can have electrodes. The stent can be collapsible, expandable, or both. The implant 122 can be delivered to the implant location using a delivery device such as a catheter. For example, FIGS. 6A-6C illustrate an implant 122 being guided from a first intermediate position illustrated in FIG. 6A, to a second intermediate position illustrated in FIG. 6B, to a deployed position at the target location (also referred to as the desired implant location) illustrated in FIG. 6C. As other examples, FIGS. 7A-7C, 8A-8C, 9A-9C and 10A-10C illustrate an implant 122 being guided from a first intermediate position (FIGS. 7A, 8A, 9A, 10A), to a second intermediate position (FIGS. 7B, 8B, 9B, 10B), to a deployed position at the target location (FIGS. 7C, 8C, 9C, 10C).

All of the images and imaging techniques disclosed herein can be used for surgeries that involve intra-operative magnetic resonance imaging, intra-operative CT, or both. For example, such surgeries can include neuroendovascular surgeries. The intra-operative CT can include contrast enhanced CT and non-contrast enhanced CT.

The variations described herein are offered by way of example only. Moreover, such devices and methods may be applied to other sites within the body. Modification of the above-described assemblies and methods for carrying out the invention, combinations between different variations, and variations of aspects of the invention that are obvious to those of skill in the art are intended to be within the scope of the claims. The above-described variations, configurations, features, elements, methods and variations of these aspects can be combined and modified with each other in any combination. Any elements described herein as singular can be pluralized (i.e., anything described as “one” can be more than one). Any species element of a genus element can have the characteristics or elements of any other species element of that genus.

What is claimed is:

1. A method for implanting a medical device within a vessel in a region of a patient's body, where the device is configured for sensing or stimulating tissue adjacent to the vessel, the method comprising:
obtaining an activity image of the region of the patient's body, where the activity image displays a neural activity in the region;
obtaining a structural image of the region of the patient's body, where the structural image displays an anatomic structure of a vascular network in the region, where the vessel is part of the vascular network;
co-registering the activity image with the structural image to produce a composite image of the region showing the neural activity displayed in the activity image relative to the anatomic structure of the vascular network of the structural image;
identifying a land-mark based target corresponding to the neural activity;
acquiring a real-time image of the region of the patient's body;
co-registering the composite image with the real-time image to select a target location within the vascular network using the land-mark based target; and
implanting a device within the target location, where the device is configure d to sense and/or stimulate neural activity in the region.

2. The method of claim 1, where obtaining the activity image comprises performing at least a first non-invasive imaging of the region.

3. The method of claim 2, wherein the first non-invasive imaging of the region comprises an imaging modality selected from the group consisting of functional magnetic resonance imaging (fMRI), MRV, electroencephalogram (EEG), magnetoencephalography (MEG), functional near-infrared spectroscopy (fNIRS), functional positron emission tomography (PET), and any combination thereof.

4. The method of claim 1, where obtaining the structural image comprises performing at least a second non-invasive imaging of the region.

5. The method of claim 4, wherein the second non-invasive imaging comprises an imaging modality selected from the group consisting of: structural magnetic resonance imaging (sMRI), magnetic resonance venography (MRV), computed tomography (CT), and any combination thereof

6. The method of claim 1, further comprising identifying the target location with a virtual marker and displaying the virtual marker on the real-time three-dimensional image.

7. The method of claim 1, where identifying a landmark based target corresponding to the neural activity comprises identifying a plurality of landmark based targets.

8. The method of claim 5, wherein the landmark based target comprises a target selected from the group consisting of a target location, a deployment location, a structural landmark, a functional landmark, a structural and functional landmark, a problem area, and any combination thereof

9. The method of claim 1, further comprising immobilizing the region of the patient's body prior to acquiring the real-time three-dimensional image.

10. The method of claim 1, wherein the activity image is three-dimensional.

11. The method of claim 1, wherein the structural image is three-dimensional.

12. The method of claim 1, wherein the real-time image is three-dimensional.

13. The method of claim 1, wherein the real-time image is two-dimensional.

14. The method of claim 1, wherein implanting the device within the target location comprises advancing the device through the vascular network of the patient.

15. The method of claim 1, wherein co-registering the activity image with the structural image further comprise overlaying the activity image with the structural image.

16. The method of claim 1, wherein co-registering the activity image with the structural image further comprise displaying the activity image and the structural image separately side-by-side.

17. The method of claim 1, wherein co-registering the composite image with the real-time three-dimensional image to select the target location within the vascular network using the landmark based target comprises displaying a virtual representation of the landmark based target on the real-time image.

18. A method for providing guidance for implanting a medical device within a vessel in a region of a patient's body, the method comprising:
obtaining an activity image of the region of the patient's body, where the activity image displays a neural activity of non-vascular tissue in the region;
obtaining a structural image of the region of the patient's body, where the structural image displays an anatomic structure of a vascular network in the region;
co-registering the activity image with the structural image to produce a composite image of the region showing the neural activity displayed in the activity image relative to the anatomic structure of the vascular network of the structural image to identify a land-mark based target;
acquiring a real-time three-dimensional image of the region of the patient's body;
co-registering the composite image with the real-time three-dimensional image such that the land-mark based target can be identified using the real-time three-dimensional image to provide an operative map to a medical practitioner such that the medical practitioner is able to use the landmark based target to identify an implant location for implantation of the medical device in the region of the patient's body.

19. The method of claim 18, where obtaining the activity image comprises performing at least a first non-invasive imaging of the region.

20. The method of claim 19, wherein the first non-invasive imaging of the region comprises an imaging modality selected from the group consisting of functional magnetic resonance imaging (fMRI), MRV, electroencephalogram (EEG), magnetoencephalography (MEG), functional near-infrared spectroscopy (fNIRS), functional positron emission tomography (PET), and any combination thereof.

21. The method of claim 18, where obtaining the structural image comprises performing at least a second non-invasive imaging of the region.

22. The method of claim 21, wherein the second non-invasive imaging comprises an imaging modality selected from the group consisting of: structural magnetic resonance imaging (sMRI), magnetic resonance venography (MRV), computed tomography (CT), and any combination thereof.

23. The method of claim 18, further where identifying a landmark based target corresponding to the neural activity comprises identifying a plurality of landmark based targets.

24. The method of claim 23, wherein the landmark based target comprises a target selected from the group consisting of a target location, a deployment location, a structural landmark, a functional landmark, a structural and functional landmark, a problem area, and any combination thereof

25. The method of claim 18, wherein the activity image is three-dimensional.

26. The method of claim 18, wherein the structural image is three-dimensional.

27. The method of claim 18, wherein the real-time image is three-dimensional.

28. The method of claim 18, wherein the real-time image is two-dimensional.

29. The method of claim 18, wherein co-registering the activity image with the structural image further comprise overlaying the activity image with the structural image.

30. The method of claim 18, wherein co-registering the activity image with the structural image further comprise displaying the activity image and the structural image separately side-by-side.

31. The method of claim 18, wherein co-registering the composite image with the real-time three-dimensional image to select the target location within the vascular network using the landmark based target comprises displaying a virtual representation of the landmark based target on the real-time image.
